SEQUENCE LISTING (2 sequences)

SEQ ID NO: 1 - DNA, 18067 bp, Streptococcus thermophilus
  misc_feature 1..18067: note = DSM33981-eps cluster
  source 1..18067: mol_type = genomic DNA
atgagttcgcgtacgaatcgtaagcaaaaacgtacgggtaatagatcgtgggggatggtcaacgttggattgaccattctgtatgctattttagcattggtcttattattcaccatgttcaattataatttcctatcctttaggtttttgaacatcattatcaccattggtttgttggtagttcttgctattagcatcttccttcagaagactaagaaatcaccactagtgacaacggttgtattggttatcttctcgctagtttctctggttggtatttttggttttaaacaaatgattgacatcactaaccgtatgaatcagacggcagcattttctgaagtagaaatgagcatcgtggttcctaaggaaagtgacatcaaagatgtgagccagcttactagcgtacaggcacctactaaggttgataagaacaatatcgagatcttgatgtcagctctcaaaaaagataaaaaagttgatgttaaagttgatgatgttgcttcatatcaagaagcttatgataatctcaagtctggcaaatctaaagctatggtcttgagtggctcttatgctagcctattagagtctgtcaatagtaactttgcttcaaatctaaaaacaatttatacttataaaattaaaaagaagaataacaactctgcaaaccaagtagattcaaaagtcttcaatatttatattagtggtattgatacctacggttcgatttcaacagtgtcacgttcagatgtcaatattattatgacagtaaacatgaatacacataagattctcttgacgactactccacgtgatgcatacgttaagattcctggtggtggggcaaaccagtatgataaattaacccacgcaggtatttatggtgttgaaacatctgaacaaactctggaagatctatatggtactaagattgattactatgcacgaattaacttcacatctttccttaagttgattgaccaacttggtggtgtgacagtccataatgatcaagctttcacaagtcttcatgggaagtttgatttcccagttggagatatccaaatgaattcagagcaagcacttggatttgttcgtgaacgctatagtttagatggcggagataatgatcgtggtaaaaaccaggaagaagtcatttctgcgattgtaaacaagttggcttctctaaagtctgtatcaaactttacttcaatcgttaataatctccaagactctgttcagacaaatatttctttggataccattaatgctttggctaatacacaacttgattcaggctctaaatttacagtaacgtctcaagcagtaacaggtacgggctcaaccggacaattgacctcttatgcgatgccaaattctagtctttacatgatgaaactagataattcgagtgtggcaagagcctctcatgctatcaaaaatctgatggaggaaaaataagtgattgacgttcactcacatattgtttttgatgttgatgatggtcctaaaactttagaagaaagtttagacctcattgatgaaagttacgcccagggggtacgtaagattgtttcaacatcccatcgtcgtaaggggatgtttgagactccagaggataaaatttttgccaacttttctaaggtaaaagcagaagcagaagcactttatccagacttaactatttattatggaggtgaactttattacacctcagacattgtggagaaacttgaaaagaatctcattccgcgcatgcacaacactcaatttgctttaattgagtttagtgctcgcacatcttggaaggaaattcatagtgggcttagtaatgttttgagagcgggggtaacgcctattgttgctcatattgagcgctatgatgctctcgaagaaaatgctgatcgtgttagagaaatcatcaatatgggctgctatactcaagtcaatagctcacatgtcctcaaaccaaagctctttggagataaagataaagtaagaaagaaacgtgttcgctttttcttagagaaaaatttggttcatatggttgctagcgacatgcataatcttgggccgagaccaccatttatgaaagatgcttatgaaattgttaaaaagaactacggcctcaaacgtgctaagaatctttttattgaaaatcccaaaacattactagaaaatcaatatttataggagatattatgaatcaagataacactaaaagtgttgaaatcgacgtactagcattgctacataaactttggacgaagaagcttttgattcttttcacagctttttatttcgctgctttcagtttcttaggtacttatttctttatccaaccaacatatacatcaacaacgcgtatctatgttgttaatcaggcaacagataataagaatctttctgctcaagatttgcaagctggtacctatttggcaaatgactataaagaaattattacatcaaatgatgtcttgtcagaagttatcaaagatgaaaaattgaatatgacagaagcagaacttgctaaaatgatttcagttgatattcctactgatactcgtcttatttcaatttctgttaatgctaaaactggtcaagatgcgcaaacacttgccaataaggttcgtgaagttgcttcagaaaaaatcaagaacgtgacaaaagttgaagatgtcacaacacttggagaagctaaattgccagagtcaccatcttcaccaaatatcaaacttaatgtgcttcttggggcagtgcttggagcattccttgcagtggttggtgtattggtacgtgaaatcctagatgatcgtgttcgccgtccagaagatgtggaagatgcccttggaatgacacttcttggaattatccctgatacagataaaatttaaggagaagaaatgcctttattaaagttagttaaatcaaaagtagactttgctaaaaagacggaagagtattataacgctattcgcacaaatattcaattttctggtgctcagatgaaagtgattgcgattagctctgttgaagctggtgaaggaaaatcaacgacatctgttaacttggcgatttcatttgctagtgttgggctccgaacacttctgattgatgcggatacgcgtaattctgttttgtcgggtacatttaaatcaaatgagccttataaaggtctttcaaatttcctttcaggaaatgccgatctaaatgaaacgatttgccaaactaatatttctggtttggatgttattgcatctggtcctgttccacctaatccaacaagtcttttgcaaaatgacaattttagacatttgatggaagttgctcgtagtcgttatgattatgtcatcatcaatacaccaccaattggtttggttattgatgcagttattattgcccatcaggctgatgctagttttttggttacagcagctgggaaaattaaacgtcgtttcgtaactaaggcggtcgaacaattggaacaaagtggttctcagttcttaggggtcgtccttaataaagttgacatgacagttgataaatatggatcgtatggttcttacggatcatatggcgagtatggaaaaaaatctgaccaaaaagaaggtcattcaagagcacatcgtcgtagaaaaggatagcattaatggggatgatgcggctccttataccttaacagattaaaaaggggtttagagtgaaagaaaaacaagaaattcgtcgcattgaaattggtattatacagttggttgtggttgttttcgcagccatggtagctagtaaaataccatatacagagattacccaaggaagtattgtccttttaggtgtcgtacatgtagtgtcttactatatcagtagttattatgaaaatcttaagtatagaggctacttggatgaactcattgcaactgtcaaatattgtttcatatttgctctaattgcaacatttctctcgttttttgcagatggaagtttttcaatctcacgtcgcggacttctttacgtcaccatgatttcaggtgttctcttatacgttacaaatactgttcttaagtatttccgctcatctatttatacacgtcgtaaaagtaacaagaatattctcttgatttctgatcaggcacgtcttgataatgttttatctcgtatgaaggacaatatggatggtaggatttcagcagtttgtgtcttggataatccttatttcactgatccatttatcaagagtgttaaacctgaaaatttgattgaatatgcgacacactcagtagtagaccaagttttgattaatcttccaagtgagcagtacaagatttggaattacgcatcaccttttgaagttatggggattccagtatccattaatttgaatgcacttgaatttatgagtaagggtgaaaagcgtatccaacaattgggtcctttcaaagttgttacgttttcaacgcatttttatagctatggagatatcttggcgaaacgtttcctcgatatctgtggagccctagttggtttggtgctctgtgggattgttggaatcttcctttatccacttattcgtaaggatggtgggccagccatttttgctcaagaccgtgtgggagaaaatggacgtatcttcaagttttataaattccgttctatgtgtgttgatgcggaagaaatcaagaagaatttgatggcacagaatcaaatgtctggtggtatgtttaagatggacaatgatccacgtattaccaaaattggacatttcattcgtaaaacgagtcttgatgaacttccacaattttggaatgttctaaaaggtgatatgagcttggtaggaacacgtccaccaacattggatgagtacgaatcttatacaccggaacaaaaacgtcgcctcagctttaaaccaggtattactggtctttggcaagtaagcggtcgaagtgaaattactgattttgatgaagttgtaaaactagacgttgcttatttggacggatggacaatctggcgcgatattaaaatcttattgaaaacaattaaagtagtagtaatgaaggatggagcaaagtgatggctttctccatttcttttaatggtgattaaatgacaaaaacagtttatatcgttggttctaagggaattccagcaaaatatggtggatttgagacctttgttgagaagttgacagagttccaacaagacaaagatatccaatattatgtagcttgtatgcgtgaaaattctgctaaatcaggaattagagcagatactttcgaatataacggtgctaaatgctacaacattgacgttccaaatatcgggcctgcacgtgccatcgcttatgacattgcagctcttaataaagccattgaaattgctaaggaaaataatgaccaagcaccgatcttttatgttttggcctgccgaattggtccatttatctcaggtattaaaaagaaaatcaaggctatcggtggtacccttatggttaacccggatggacatgaatggcttcgtgcaaagtggagtcttccggttcgtaagtattggaaaatttcagagcaactcatggttaaacatgccgatctcttgatttgtgatagtaaaaacattgaatcttatattcaaaatgattatgcgaaatatcaacctcaaacaacctatatcgcctatggaactgatacaactaaatctcgattgactaaggttgatgataaggtacgcacatggtattttgaaaagggggtcattgaaaacaattattaccttgttgtaggacgcttcgtgcctgaaaataactatgaggctatgattcgtggctttatggcatctaactctgacaaggactttgttctcgttaccaatgttgaagaaaacaagttctacgacaagttgcgtcaggaaactggttttgacaaggatcctcgtgtgaagtttgtcggaactgtttatgaccaagaattgctcaagtacattcgtgagaatgcctttgcttatttccatggtcatgaggttggtggaaccaatccatcacttcttgaggcacttgagtcaactaagctcaatctcttgttggatgttggctttaaccgtgaagtcggtgaagatggtgctctctattggaagaaagaacaattggcaagtgtcattgatcaagctgagcgattggatgaagaaactatcgcagacctcaaccaaaaatcaagtaatcgtatcgcagaagcctttacttgggaaaaaattgttatagactacgagaaagtatttaaaggatagaaatgcagaaaattttatatcttcacgctggtgccgaaatgtatggtgctgacaaggttttgttggagcttattaagggtttggataaagatgcctttgaggctcatgttatcttgcctaacgatggtgtcttagtggatgcattgaaagaggttggagctaaagtaaaagttatcgattatccaatcttgcgccgtaaatattttaatcctaagggaattcttgagtattttggctcttataatcgttattcaaagcaaattgctaaatatgctaaggataatggcattactcttattcacaataatacgactgcggtacttgagggaatttatctcaaacgtaagttgaaacttcctttgatttggcatgttcacgagattatcgtcaaaccaaaagcaatctctgatttcatcaactttttgatggggtgttatgctgatacgattgtgacagtttcaaatgctgtggccaaccacgtcaagcagtctcgctttgtaaaaaatgaccaagttcaggtcatctataatggtgttgacaatgccgtctatcatgaaatggatgccagcgcagttcgtgaccagtttggtatcgcacaggatgccttggttatcggaatggttggtcgagtgaatgcatggaaaggtcaaggcgatttcttggaagccgtgactccaatcttaaaagctaaaccaaaagcagtagcctttctggcaggtagtgcttttgaaggtgaagaatggcgagttgatgagttggaaaaggctatatcagactcaccagtagctgaacaaatcaaacgtatcgattattatagcaagacgacagagctctataatctgtttgatatctttgtcttaccaagtaccaatccagatcctctaccaactgtagttctcgaatcaatggcttgtggcaaacctgtagtcggctaccgtcatggtggtgtctgtgaaatggttaaagaaggcgaaaacggccttcttgccacaccaaatcagcctgcagaattgtctaaggccatccaagaattagctgataacactgagaagagagagcaatttggcaaggcctctgtcaaacgccaaaaagaactcttctcattacaaagttatattcgtaatttctcagaactatataaaaaataaaaagaagtattaagaagtactagtatgaacagcgaaacactaaaaaataaattaaaaccaatcgtttatccaattattaattttattcctagacgaagacttaagaataaaaattttactatcatttgtgataattgttgggctggaaaagtctatcaggaattgggcttaccttatcaaacgccatttgtagggatgtttgttttctctcctgattacatcaagatgctcaagaatttaaaacactatttgagtggaaatatccccttgaaatttgttaaaaaatcgaaatacattaaagactttgataatgcttatccacttgcacttcttgacaatattgaacttcattttttgtattatgcagatgaagaagaagctactcaaaaatggaatcgtcgtttagaaagaatacattgggataatttgtatttcaagtttaatgataatgatgcctgcacttatgagctgatgaaagagtttgaagaacttccatacaagagtaaggttattttctcttcgaagaattacagtgacctaccttcattggtccactttaaatcagctgagaagcaaggacatgttggaattgatttaaaaacttaccaccgctattttaatgctgttacctggttgaataagggtggtgaagatttaacataaaataaaaatttaggtgaagaatccatgaaaccacttttaactgttgtcattccagtgtataatgtcgaaaagtatttaaaacgttgtattgagagtatccttattcaagagtggaagaactatgacatcttacttgtagatgatggaagtactgatagttctcctcaaatctgtgatgattatgctaaagtttatgattttatctcagtaattcataaaaaaaatggaggtctttctgaagctcgcaatactggtatttcacatgctaagggtgactatgtttatttccctgattctgatgactggcttgaaccacaaacatttaaagaattagcagacgtattggaatcccaagagtttgatatcgtttcttttaaccgtgagtttgtgaatggtgaagaagatcctatagtttcagatccattagtaactcaagtgtatgaaggtaaagatgcttttgtccaaatgctcaagcaaagttatatcacaggttttgccaacgacaaaatttataaaaagtctctatttattgataataatatttcttttccaaaaggaaaatattatgaagacttgggaacgaattacaagctttttctttcagctgagaatgtctttgctaccaatcaaaagtattatcactatttaattgataatccggattccattacacaatcttggaatgagcagaagtttagtgatatgtttgaattttataaagatattttctattctgattttgttcgttcacgattaaatcaagaagaattacaaatttcacagctatattatgtgaatggtttgactcacatcttagcaagtttatataaatctaaattagataaagaatatattgacattactaatgaagtgaaacaagaattacttaagaatagtgtctctctttctcaaatgaaagaccaaccaaataagttaaaatatatactgtttagacttaaaatattaaaaccagccttcagtattcaaagtattctctaatatgatttaggagttttatttacattctttattcagagttttaggttatgaagtctaaactatttatttttgataatgggatacaaagcgttggtcaaatttgaataacttggcttgaaaaatatttaataggtaatttaacatggaaaaaaattgatttcgttgtactttgggttgatggaaacgatccagaatttattagagaaaaaaataaatacacacctcacaataagaaaatcgataatgatgaagataatgtgcaccgttatcgtgactatggcacattcaattattggtttcgaatggtagagaggcatgcaccgtgggtaaataatatttatcttattactaacggtcaaagaccgaaatggttaaattgtaaatcatcctaaacttaaatgggttagacatgaagaatttattccaaaggaatatcttccaacattcaatgcttctgcattgagatgaatatacatcggatagatggtttatctgaaaatttcgttctgtttaatgatgatatgtacctaatccaagatgttaaatatagtgattttttttgtcaatgaaaaacccaagttgttagctgtttatgaagccttagttccatggtctcgtttttcaaaaatttattttaatgatgtacttgttctttataggcattttccaaataaaaaagccttgagacagtcaccatttaagttttttaatattaaatatggccaattgatgctaaaaaataggctccataatttccatggtgggtttacccactacagaaattatagagccaaaatcggaagacatatttggtttttcgaaggaaattttttattcacaagcggtacaaagtgttttcaattcatctgagttaaaaatattgaattgtttttatacagataatcttatctatgttcttacaattttatataaaacgaatttatcagaagagtgtaagcaactaactatggatgttaattcagaactaaggaagaatggagtatcgttattaaggttcaaaaaacgtataaatttcaagaaatatgtgctgtacaagctgggcttgttgaagttagtttttgacttacgattaagaatgaagagataccgatagaaatattatgagaaaaacactactactactggagcaaattgattcctttgagcaattttttgcgaaaagtgtgtcgcttcctcaagtcgaggtttcaacaccttataagaaagttcattcacagatggcgcacaaaatacgggctatatcaaaacgtctaccgtttgcttctttacatcaattttggattgcagagtggcaaaaacgaatagatgattttgacactatagttgtttttgataatgcagtatcgcagaaactattagaatttattaagtctcggataacgaagcgaacaaaattaaaaatttggctttggaatgttcccaatgagagaattgaatatttgaaagaaaattttgatgtttattgttttgatcagaattattcaaaggagtatgggctaacctttttggaacagttttacattttggatagtgttaatcttgatgaaaaaacagatgtcttgcgagatttttattttgttggagctgataaaggtcgtatacagcaactggatcaattagctggaatgatagaggaatcagagttgacctatcagtttgatgtttttagtgacgataaacagtcgaatcgtagtggaataaattatcttgatagtaaaattacctaccctgaaattatcgagaaaattcaagaatcaaaggtgattgtagaaattaataaagagggacaagtgggtctaactttacgggcattggaagctattttttatggtaaaaaattagttactaacaattccaatatcaagaagtatgatttctacagtcctaacaatattcttgtgctaggtgatgaaagctctttggatttgatagaatttttaaacaaaccatatcagattttgccgaaacacattattgcaaagtactcattcagtaattggttgaatcgtatgacaaaatttaataggagagaaaaatgaaagtgactttctgccttcctgaaataagtcaagttcctatgggaggttataaaattatttttgaatatgcaaatcgattgcaaaaaaggggacacaaggtaactattgtctttttaacacataatgtgtggagtcgactaaccaataacaagaagattaaatctatcgtagggaagattagaggacggaatgaaccatcgtggtttcatttaaattctgaaatacaaaaaataatgacaccttatttagatggtcgagagtttcctgacgcagattttatttttgcaactgcagtaacaacagctaagattgttaaagaattaccatctcgttgtggtaaaaaatgttattttattcaaggttttgaaacatttatccttccagaggatgaagttgttgaatcttatcaatatggttttctaaatttcacagtttcaaattggttgagtaatattgtgggagcgtatacaagtgataaaacaatctgccttccaaatcctatagatactgaaaattttaaagtgataactcctataaacgaacgtaatccttacacattggggatgctttatcatgaaggggagcataaaggaattccctacgctttagaagccattgatatagtcaaaaaaaaatataaggatgttgtggttaacatttttggtgtaccgaatcgtccagaatcgttacctgattattttagatatgtacagaaagcaaatcagaatgatttattgagcttatataatgctacttcaatttttatttgtgcaacaattgatgagggctttggtttaacaggcgctgagagtatggcttgtggttgtgcattagtttctacagcttataatggtgttcttgaatatgcagtaaatgccgaaaatgctttattgtctcccgtgagagatagtaaagcattagctgaaaacatcataaaacttatcgaaaatgacccttatcgtcagtcgttagctaaaaaagctagcgaatctataagtgaaagatcatgggataaaacaattcaaaaattagaaacagttttggcaagtgaactagatatgggtagaggttaatatgattagaatagagagagtgataaatttctttttttatatcaatgttgttctgtacatgttttctttgtctaacattccatttttatttcatatggagtttataaatgttataccaactgttttaggattactagcttatatgatgtactattataaggcaagaaaacttccaactaattcaatgccatctttactattgctacttttgtataccttctttgtagtattgttttggaaaaaatttgatatcgattggtcgctagtaagtatattaatatatgttccattaattgagtctgaaaataagggctttatccgtgggcttatacttgttaaattatttgtattaatgattgttgtagtattgtcacttcttggaataataactgataccgtttacttgaaattaggtggtgtcagtcattcacttggtttttttcatcctaatacgttaggagcagtcagtttatctattttctttgactgttttatattatttcaagagttgagaaaacaatattttacattttttcttcttacagtattatacatagtctatcacctaacattttcaagaacatcaattattatagctggactaggattattaatttatgcattccgtaagatattgtcgagttatgtaatgaataagtttgttttctcgattactactactggtgtgtatctttttggaatgttttactcaatgttttatagcacttccaacatcttttataggcaattagatatatttctaactaatagagttcgcctgggaaatatgtatattcaaagatatggttttacactttttccaaaaagtactcccaatatcatacctcaaggatggtggagttcagaccaactttttaatgataatacatttctgaaatttgcacttacacagggagtaattattgctgtattatttctgctatatattttatacattataggaactaaagagtattgcttgtacgatgcgttgcttattttactaacgtttttattcttaatgatcgaagctctaggattttacgccttcttattgacaccattactcttcaagttcatggctgttagagatagtagtgtgaagcaaatataaaaattatcataacatgtttatgcgataaaggtgatttatgaaaacagtaaaaaactatgtttatagttcattatatcaattatttttaatcattgttccatttgcaacaattccttatgtatcacgtgtgttgggtgctgagttgataggtgtcaattcgtttacgaacacaatcatgacatattttgtactattcgcaaacttaggaacaactgtttatggaaatcgaacaattgcttattaccgagaatcaataacgaagcgaagtcaaaagttttgggaaatccttattctaaaattggttgtttcattagttatctacctcatattcttagtttttatttggttataccctaactatagaaccgtttttatgatacaatcagtccaaattttggctacagcagttgatgtctcatggctgtttgatggattggaagatttcaaacgtacagttgttcgtaattttatcgtgaaattggcatctgtaatgatgattttcttgtttgtaaaaactcctcaggattttaatacatatgttttaatcatggtggggtcgtctttagtggggaatctagctctttggacatatttaagacgctatattacaagtattaagtttactaatttgaatatagcagagcattttgcaccagtgttcgctttatttgttccacagattgcctctactatttttgtttccttaaataaagttttgctaggaactctatcaacgatgtcacaggcaggttattttgaaaatgctgataaggtaataagagttttactggcgttagtttcgtctattggtgtagttatttttccaaaggttgctaatgcctatatgaatcgagacactaagaaagtgatagaactgacaaagttaacttttaatgctgttaatatcattacaattcctatggtaattgggatgatatcaattagtgatattttttcggatatcttttttggaccggagtttttgggaattgatattgtattgtcaattttaattctagaactattatttatgggatattcttcagtgttgggaagtcaatatttgattgctacaggacaggcaaaatatttaagtgttgctgtgattggcggtctttttactactagtgtatcgtctattattctgattcctcatcatggtgctgttggggcagcaattgcgtcagtggttggagaggctactatagcgttcatcgagcttatttttatcagaaagcaaatcaatatattagagttatgtaaggacgtccctaaatatagtatagcaagcctattaatgtttttaggaatctatatgacaaagttactcacagtccctccctatataaatttgtttatgagaattatagtaggcggggttatctatggcgtaagtttattgttgctgaagccacaattggtgaaaatttctattgagaaaataaagtttgctaagaaataaatggaaagcagtgcgacgataagaaacgattgtttagattaattttacaaattctgtttaaataaaatatcataatctcaaaactatgggaatagcaaccttaggaattatatcagaatttgcccttatgatggcttagggaaaatatattgacagggagcttagctccctttttcaaatgacactaatgtttaaacatggtaaaatagacttaataaatttttgattaaaaggtgtcataacatgaaaggtattattcttgcaggtgggtctggaactcgcctttatccactgactcgtgcggcgtcaaaacagttgatgccaatctatgataagccaatgatttactatccgctttcaactttgatgttggctgggattaaagaaatcttgattatttcaactcctcaggatcttcctcgtttcaaggatatgctgggagacggctcagagcttggtatcaccttgtcatacgcagaacaaccaagcccagacggcttggcacaagcctttatcatcggtgaagcctttatcggtgatgacaacgtagccttgattttgggcgacaacatctatcatggaaatggtttgaccaagatgttgcaacgagctgctagcaaagaaaaaggtgcaactgtctttggttaccaagttaaagacccagaacgttttggtgttgtcgagtttgacgcagacatgaacgctatctctatcgaagagaaaccagaagagccaaaatcaaactttgcggttactggtttgtacttctatgacaatgatgttgtggagattgctaaaaacatcaaaccgtcacctcgtggagagttggaaatcaccgatgtcaacaaggcttacttagaccgtggcgacttgtctgtggaactgatgggacgcggattttcttggttggatacaggtacgcatgagagcctcttggaagctgctcaatacatcgaaacggttcaacgtttgcaaaacgttcaagtggcaaacttggaagaaattgcctaccgcatgggctacatcacaaaagaacaagtgtacgaattggcacagccactcaagaaaaatgaatacggacaatacttgctccgtttgattggagaagcttaatgacagaaaactttttcggaaaaacactcgcagcccgtccagttgaaggtattccaggaatgttagaatttgacattccagttcatggcgacaaccgtggctggttcaaggagaacttccaaaaggaaaaaatgtggctccttgtcaactgtagtgggtgacgaaaagctaacatctagagaggaccggataggttttctctttttgtatgttcagagtgatgaagacacgcttcctaaagttgatgaagtttctaaaaccaaagaccaaccgtttgatgtctttaatcaacttgttagtcgcttcgagttttgcgtttgaatagtccgtttctagtgcgtttttgatggatttcttgtgtctaagaaaagtcctaaagaccgtttgaaagtagtggttgaccttgcttatgttctcctctattaactcaaagaactaatctactctcttctcctgaaagtgaaaaagtaaaagctgataaagtttgtaggcattagcgatctcttctgaaaataccaatgtcttctcaatgatttcatgtggtactaaagtttgtctgaaggtctttgaatagaaggagttgagagatagcttacgactgtccttttgaaaaagtcgccagtgattttttaaatctcaataaggtagtgaattcttattaaactggttcataatggcaattcttgtctttaaaaaggcacgtccaaggtgctggatgatgtggaaacgatcaagaacgatttttgcgtttggaaagagcttgcgagccagtggaatataagctccagacatatccatcgtgataaattgtacctgttgtcggactttcaatggatacttcaaatagtagtttcgtatagtagtttggcggcgattatcaaggatgattatgagttcgtttgtctcatacttttgagctacaaaagccaactctcctttcttgaaaccaaactcatcccaggacataacagcagggagtttgtcataatgtttcttgaaagtaaactgatcaagcttacgatagacagtggacgtcgacacgcgaagtcttcttgcaatatcagttagtgacaccgtctcagttaggagttgtgtaaccttttgtcggaccagattagagatttggcagtttttctcaacgatagatgtctcagctaccgtgactctcctacaatttttatactggaaacgacgttttttcagacgtagtagagttggtgttcccgcttgcttgagaagagggattttagatttttttgaaagtcatatttgatcatctttccttggcagtgaggacatgatggtgaagggtaatcaagttttgcttgaatctcgatatgagtgtcagtttcaaaaacaagtgaaataatgatgttttggtctttgattccgattaattctgtggtattcttaataggtctcataagttcttcctaatgatggtttggttgcttttcattatagttcttatgggactttttgtgtacactcaaaaagctctataatctctacagtagttttacccactacagaaattatagagccaatatatctcctgtctatttttatgctacttttgggttagctcaactcaaccgccttttaatctcccaacaacaataatacccaatcaaacaacccaaaaaattcaagataatatcactaatggcaaatgtgcccaaataaaagataaattgaatggtttcaattcctaaaagagtgaccaaactgacaatgacaaactgtttgaaatcagtattgatacagtaaaggccacctaaaggaatgaagtagataatatttagcacagcctcttgaatcgttctggtatccgcttttataaagtcaaaaggattcagtgatatcgcctgaaaatccgttgttttagtaaaaagcaccatgaataacagtaataaatacacactgaaagcaagatagagataaataactgaaaaatgtttgaggtgatactggatgccaaacaaccagataatcagcgttaataagagtattaaagttaatgtggtatagtcaaagtggttaatcaacctagtcaggctttgatagcgagtgagaacgggcataatcagccaagtaatcgtcgcataactcaggataaatgtgaccaataaactgctgaggtagatcatatattttcgcaa

SEQ ID NO: 2 - DNA, 20467 bp, Streptococcus thermophilus
  misc_feature 1..20467: note = DSM33982-eps cluster
  source 1..20467: mol_type = genomic DNA
atgagttcgcgtacgaatcgtaagcaaaaacgtacgggtaatagatcgtgggggatggtcaacgttggattgaccattctgtatgctattttagcattggtcttattattcaccatgttcaattataatttcctatcctttaggtttttgaacatcattatcaccattggtttgttggtagttcttgctattaacatcttccttcagaagactaagaaatcaccactagtgacaacggttgtattggttatcttctcgctagtttctctggttggtatttttggttttaaacaaatgattgacatcactaaccgtatgaatcagacggcagcattttctgaagtagaaatgagcatcgtggttcctaaggaaagtgacatcaaagatgtgagccagcttactagcgtacaggcacctactaaggttgataagaacaatatcgagaccttgatgtcggctctcaaaaaagataaaaaagttgatgttaaagttgatgatgttgcctcatatcaagaagcttatgataatcttaagtctggcaaatctaataaagctatggtcttgagtgggtcttatgctagcctactagagtctgtcgatagtaattatgcttcaaatctaaaaacaatctatacttataaaattaaaaagaagaataacaattctgcaaaacaagtagattcaaaagtcttcaatatttatattagtggtattgatacctacggttcaatttcaacagtgtcacgttcagatgtcaatatcattatgaccgttaacatgaacacacataagattctcttgacgactactccacgtgatgcatacgttaagattcctggtggtggggcaaaccagtatgataaattaacccacgcaggtatttatggcgttgaaacatctgaacaaactctggaaaatctttatggtattaagattgattactatgcccgaattaacttcacatctttccttaagttgattgaccaacttggtggtgtgacagtccataatgatcaagctttcacaagtcttcatgggaagtttgatttcccagttggagatatccaaatgaattcagagcaagcacttggatttgttcgtgaacgctatagtttagatgacggagataatgaccgtggtaaaaaccaggagaaagtcatttctgcgattgtaaacaagttggcttctctaaagtctgtatcaaactttacttcaatcgttaataatctccaagactctgttcagacaaatatttctttggataccattaatgctttggctaatacacaacttgattcaggctctaaatttacagtaacgtctcaagcagtaactggtacaggttcaaccggacaattgacctcttatgctatgccaaattctagtctttacatgatgaaactagataattcgagtgtggcaagagcctctcaagctatcaaaaatctgatggaggaaaaataagtgattgacgttcactcacatattgtttttgatgttgatgatggtcctaaaactttagaagaaagtttagacctcattggtgaaagttacgcccagggggtacgtaagattgtttcaacatcccatcgtcgtaagggaatgtttgagactccagaggataaaatttttgccaacttttctaaggtaaaagcagaagcagaagcactttatccagacttaactatttattatggaggtgaactttattacaccttagacattgtggagaaacttgaaaagaatctcattccgcgcatgcacaacactcaatttgctttgattgagtttagtgctcgcacatcttggaaagaaattcatagtgggcttagtaatgttttgagagcgggggtaacgcctattgttgctcatattgagcgctatgatgccctcgaagaaaatgctgatcgtgttagagaaatcatcaatatgggctgctatactcaagtcaatagctcacatgtcctcaaaccaaagctctttggagataaagataaagtaggaaaaaaacgtgttcgttatttcttggagaaaaatttggttcatatggttgctagtgacatgcataatcttggtccaagaccaccatttatgaaagatgcttatgaaattgttaaaaagaactacggctccaaacgtgctaagaatctttttattgaaaatcccaaaacattactagaaaatcaatatttataggagatattatgaatcaagataacactaaaagtgatgaaatcgacgtactagcattgctacataaactttggacgaagaagcttttgattcttttcacagttttttatttcgctgctttcagtttcttaggtacttatttctttatccaaccaacatatacatcaacaacgcgtatctatgtggttaatcaggcaacagataataagaatctttctgctgaagctttgcaggccggtacatttttgacaaaagactacaaagaaattattacatcaaacgatgtcttgtcagaagttatcaaagatgaaaaattgaatatgacagtagcagaacttgctaaaatgattttagttgataatcctactgatactcgtcttatttcaatttctgttaatgctaaaactggtcaagatgcgcaaacacttgccaataaggttcgtgaagttgcttcagaaaaaatcaagaacgtgacaaaagttgaagatgttacaacgctcgaagaagctaaattgccagagtcaccatcttcaccaaatatcaaacttaatgtgcttcttggggcagtgcttggaggattccttgcagtggttggtgtattggtacgtgaaatcctagatgatcgtgttcgccgtccagaagatgtggaagatgcccttggaatgacacttcttggaattgtccctgatacagataaaatttaaggagaagaaatgcctctattaaagttagtaaaatctaaagtaaactttgccaaacaaacagaagagaattacaatgccattcgcacaaatattcaattttctggtgctcagattaaagtgattgcgattagctctgttgaagttggtgaaggaaaatcaacgacatctgttaacttggcgatttcatttgctagtgttgggctccgaacacttctgattgatgcggatacgcgtaattctgttttgtcaggtacatttaaatcaaatgagccttataaaggtctttcaaatttcctttcaggaaatgccgatctaaatgaaacgatttgccaaactgatatttctggtttagatgttattgcatctggtcctgttccacctaatccaacaagtcttttgcaaaatgacaattttagacatttgatggaagttgctcgtagtcgttatgattatgtcatcatcgatacaccaccaattggtatggttattgatgcagttattattgcccatcaggctgatgctagttttttggttacagcagctggaaaaattaaacgtcgtttcgtaactaaggccgttgaacaattagaacaaagtggttctcagttcttaggggtcgtccttaataaagttgacatgacagttgataaatatggatcttatggttcttacggatcatatggcgagtatggaaaaaaatctgaccaaaaagaaggtcattcaagagcacatcgtcgtagaaaaggatagcattaatggggatgatgcggctccttataccttaacagattaaaaaggggtttagagtgaaagaaaaacaagaaattcgtcgcattgaaattggtattatacagttggttgtggttgttttcgcagccatggtagctagtaaaataccatatacagagattacccaagaaagcattgcctttttaggtgtcgtatatgtagtgtcttactatatcagtagttattatgaaaatcttaagtatagaggctacttggatgaactcattgcaactgtcaaatattgtttcatatttgctctaattgcaacatttctctcgttttttgcagatggaagtttttcaatctcacgtcgcggacttctttatgttactctgatttcaggtgttctcttatacgttacaaatactgttcttaagtatttccgttcatctatttatacacgtcgtaaaagtaacaagaatattctcttgatttctgatcaagcacgtctagaaaatgttttgtcttgtatgaaagacaatatggatggtaggattacagcagtttgtgtcttggataatccttattttaccgatccatttatcaagagtgttaaacctgaaaatttgattggatatgcgacacattcagtagtagaccaagttttgattaatctgccaagtgggaagtataagatttgggattatgcatcaccttttgagatcatgggaattccagtttctattaatttgaatgcccttgaatttatgagtcaaggtgaaaaacgtattcaacaattgggtcctttcaaagttgttacgttttcaactcaattttatagctatggagatatcttggcgaaacgtttcctcgatatctgtggagccctagttggtttggtgctctgtggaattgttggaatcttcctttatccacttattcgtaaggatggtgggccagccatttttgctcaagaccgtgtgggagaaaatggacgtatcttcaagttttataaattccgttctatgtgtgttgatgcggaagaaatcaagaaggatttgatggcacagaatcaaatgtctggtggtatgtttaagatggacaatgatccacgtattaccaaaattggacatttcattcgtaaaacgagtcttgatgaacttccacaattttggaatgttctaaaaggtgatatgagcttggtaggaacacgtccaccaacattggatgagtacgaatcttatacaccggaacaaaaacgtcgcctcagctttaaaccaggtattactggtctttggcaagtaagcggtcgaagtgaaattactgattttgatgaagttgtaaaactagatgttgcttatatggacggatggacaatctggcgcgatatcaaaatcttattgaaaacaattaaagtagtagtaatgaaggatggagcaaagtgatggctttctccaattttttaatggtgattaaatgacaaaaacagtttatatcgttggttctaagggaataccagcaaaatatggtggatttgagacctttgttgagaagttgacagagttccaacaagacaaagatatccaatattatatagcttgtatgcgtgaaaattctgctaaatcaggaattagagcagatactttcgaacataacggtgctaaatgctacaatattgatgtcccaaatatcgggcctgcacgtgccatcgcttatgatatcgcagctattaataaggccattgagattgctaaggaaaataatgaccaagcaccgatcttttatgttttggcctgccgaattggtccatttatctcaggtattaaaaagaaaattaaggctatcggtggtacccttatggttaacccggatggtcatgaatggcttcgtgcaaagtggagtcttccggttcgtaagtattggaaaatttcagagcaactcatggttaaacatgccgatctcttgatttgtgatagtaaaaacattgaatcttatattcaaaatgattatgcgaaatatcaacctcaaacaacctatatcgcctatggaactgatacaactaaatctcgattgactaaggttgatgataaggtacgcacatggtattttgaaaagggggtcattgaaaacaattattaccttgttgtaggacgcttcgtgcctgaaaataactatgaggctatgattcgtggctttatggcatctaactctgacaaggactttgttctcgttaccaatgttgaagaaaacaagttctacgacaagttgcgtcaggaaactggttttgacaaggatcctcgtgtgaagtttgtcggaactgtttatgaccaagaattgctcaagtacattcgtgagaatgcctttgcttatttccatggtcatgaggttggtggaaccaatccatcacttcttgaggcacttgagtcaactaagctcaatctcttgttggatgttggctttaaccgtgaagtcggtgaagatggtgctctctattggaaaaaagaccaattggcaagtgtcattaatcaagctgaacaattggatgaagaagctatctcaggcctcaatcaaaaatcaagtaaacgtatcgaagaagcctttacttgggaaaaaattgttacagactacgagaaagtatttaaaggatagaaatgcagaaaattttatatcttcacgctggtgccgaaatgtatggtgctgataaggttttgttggagcttattaaaggattggataaagaagtctttgaggctcatgtcatcttgcccaacgatggcgtcttagtgggtgcattggaaaaggttggtgctaaggtaaaagttatcgattatccaatcttgcgccggaagtattttaatcctaagggaattcttgagtattttggctcttataatcgttattcaaagcaaattgctaaatatgctaaggaaaatggcattactcttattcacaataatacgactgcggtacttgagggaatttatttcaaacgtaagttgaaacttcctttgatttggcatgttcacgagattatcgtcaaaccaaaagcaatctctgatttcatcaactttttgatggggtattatgctgatacgattgtgacagtttcaaatgctgtggccaaccacgtcaagcagtctcgctttgtaaaaaatgaccaagttcaggtcatctataatggtgttgacaatgccgtctatcatgaaatggatgccagcgcagttcgtgaccagtttggtatcgcacaggatgccttggttatcggaatggttggtcgagtgaatgcttggaaaggtcaaggcaatttcttggaagccgtgactccaatcttaaaggctaatccaaaagcaatagcctttctggcagggagtgcttttgaaggtgaagagtggcgtgttgatgagttggaaaaggcaatatcagactcaccagtagctggacaaattaagcgcatcgattattatagcaagacgacagagctctataatatgtttgatatctttgtcttgccaagtaccaatccagatcctctaccaactgtagttctcgaatcaatggcttgtggcaaacctgtagtcggctaccgtcatggtggtgtctgtgaaatggttaaagaaggcgaaaacggccttcttgccacaccaaatcagcctgcagaattgtctaaggccatccaagaattagctgataacactgagaagagagagcaatttggcaaggcctctgtcaaacgccaaaaagaactcttctcattacaaagttatattcgtaatttctcggagttatataagaaataaaaagaagtactaagaagtactaagaagtactaagaagtactaagaagtaataatatgaacagcgaaacactaaaaaataaattaaaaccaatcgtttatccaattattaattttattcctagacgaagacttaagaataaaaattttactatcatttgtgataattgttgggctggaaaagtctatcaggaattgggtttgccttatcaaactccatttgtagggatgtttgtcttttctcccgattacataaagatgctcaagaatttaaaatactatttgagtggtaatatccctttgacatttgttaaggaatcgaaatacatcaaagactttgataatgcctatcctctagcacttcttgatgatattgaacttcatttcttgcattatgcagatgaggaagaagcaactcaaaaatggaaccgtcgtttagaaagaattcattgggataatttgtatttcaagtttaatgataatgatgcctgcacttatgagttgatgaaagagtttgaagagcttccatacaagagtaaggtgattttctcttcgaagaattacagtgatctaccttcattggtccatttcaaatcagctgaaaagcaaggacacgttggcatcgacttgaaaacgtatcatcgctattttaatgttgtaacttggctgaataaaggtggagaggatctaactaaatgaaaaagataagactaattattccatattttggaaaacttccaaccttttttccttattttttgttgactgctaaacgtaatcaaaaaattgatttcttaatttatacggatcaaaaggttgaccaatttgaaattttaaatgctaaaaatatagaatttgtaactctttcttttgatgatttaagagaaagaatccagtctaagtttgattttaaaatttctctcaaaacaccttacaaattatgtgactatagggttgcttacgggcttatttttgaagaagaattaaaagagtatgattactggggattttgtgatacagatgtcttacttggtgatatttatcaatttctggaaaagcatagcttttttgagaatgattatgctcgatatggtctttttggtcacctccaaatatttaaaaattcacaagaagttaatcgtatatttatgtctggtcaaggttcaaactatcgattggattatcaaaatgtcttcactaatgagcaaagttttatttttgatgaggcaaacggtattcaaaaattgtttgaaaaatctggttttgaacaattacaagataagttttttgatgatattgatatcaggcatttttcatttcgagaatatggagaagatgagcccaaacgttattattcttggtcacagaaacatggcttgaaatcgattaatttaattgatggtaaaatagttatcaaacatccgctatatacacattttcaaaaaagaacaattgaatgtcctgaatttgaacttgtagaatcattttatgttatcccgaataagttggtatttggagaagaactttcgatgcaagaaatcgaagaagtcactaaaaataaattctattgggaatatgtcaagtcaacgttttttaaaaaatttaaaatggaaaaatggtcgttagaatttattcgccataaattaagaatgaaaaaatgaaaaaagatgtacttttaactgtcgtcattccagtttataatgtcgaaaaatatttaaaacgttgtattgagagtatccttattcaagagtggaagaactatgacatcttacttgtagatgatggaagtactgaaagttctcctcaaatctgtgatgattatgctaaagtttatgactttatctcagtgattcataaaaaaaatggaggtctttctgaagctcgcaatactggtatttcacatgctaagggtgactatgtttatttccctgattctgatgactggcttgaaccacaaacatttaaagaattagcagacgtgttggaatcccaagagtttgatatcgtttcttttaaccgtgagtttgtgaagggtgaagaagatcctatagtttcagatccattagtaactcaagtgtatgaaggtaaagatgcttttgtccaaatgctcaagcatagttatatcacaggttttgccaacgacaaaatttataaaaagtctctatttattgataataatatttcttttccaaaaggaaaatattatgaagacttgggaacgaattacaagctttttctttcagctgagaatgtctttgctaccaatcaaaagtattatcactatttaattgataatccggattccattacacagtcttggaatgagcagaagtttagtgatatgtttgaattttataaagatattttctattctgattttgttcgttcacgattaaatcaagaagaattacaaatttcacagctatattatgtgaatggcttgattcatatcttggcaagtttatataaatctaaattagataaaaaatatatagacattactaatcaagtgaaacaagaattacataagcatggtgttgctctttctcaaatgaaaggccaaccaaataagttaaaatatatactgtttagacttaaagtattaaaactagccttcagtattcaaagtattctctaatatgatttaggagttttatttacattctttattcagagttttaggttatgaagtctaaactatttatttttgataatgggatacaaagcgttggtcaaatttgaataacttggcttgaaaaatatttaataggtaatttaacatggaaaaaaattgatttcgttttactttgggttgatggaaacgatccagaatttattagagaaaaaaataaatacacacctcacaataagaaaatcgataatgatgaagataatgtgcaccgttatcgtgactatggcacattcaattattggtttcgaatggtagagaggcatgcaccgtgggtaaataatatttatcttattactaacggtcaaagaccgaaatggttaaattgtaaatcatcctaaacttaaatgggttagacatgaagaatttattccaaaggaatatcttccaacattcaatgcttctgcattgagatgaatatacatcggatagatggtttatctgaaaatttcgttctgtttaatgatgatatgtacctaatccaagatgttaaatatagtgattttttttgtcaatgaaaaacccaagttgttagctgtttatgaagccttagttccatggtctcgtttttcaaaaatttattttaatgatgtacttgttctttataggcattttccaaataaaaaagccttgagacagtcaccatttaagttttttaatattaaatatggccaattgatgctaaaaaataggctccataatttccatggtgggtttacctactacagaaattatagagccaaaatcggaagacatatttggtttttcgaaggaaattttttattcacaagcggtacaaagtgttttcaattcatctgagttaaaaatattgaattgtttttatacagataatcttatctatgttcttacaattttatataaaacgaatttatcagaagagtgtaagcaactaactatggatgttaattcagaactaaggaagaatggagtatcgttattaaggttcaaaaaacgtataaatttcaagaaatatgtgctgtacaagctgggcttgttgaagttagtttttgacttacgattaagaatgaagagataccgatagaaatattatgagaaaaacactactactactggagcaaattgattcctttgagcaattttttgcgaaaagtgtgtcgcttcctcaagtcgaggtttcaacaccttataagaaagttcattcacagatggcgcacaaaatacgggctatatcaaaacgtctaccgtttgcttctttacatcaattttggattgcagagtggcaaaaacgaatagatgattttgacactatagttgtttttgataatgcagtatcgcagaaactattagaatttattaagtctcggataacgaagcgaacaaaattaaaaatttggctttggaatgttcccaatgagagaattgaatatttgaaagaaaattttgatgtttattgttttgatcagaattattcaaaggagtatgggctaacctttttggaacagttttacattttggatagtgttaatcttgatgaaaaaacagatgtcttgcgagatttttattttgttggagctgataaaggtcgtatacagcaactggatcaattagctggaatgatagaggaatcagagttgacctatcagtttgatgtttttagtgacgataaacagtcgaatcgtagtggaataaattatcttgatagtaaaattacctaccctgaaattatcgagaaaattcaagaatcaaaggtgattgtagaaattaataaagagggacaagtgggtctaactttacgggcattggaagctattttttatggtaaaaaattagttactaacaattccaatatcaagaagtatgatttctacagtcctaacaatattcttgtgctaggtgatgaaagctctttggatttgatagaatttttaaacaaaccatatcagattttgccgaaacacattattgcaaagtactcattcagtaattggttgaatcgtatgacaaaatttaataggagagaaaaatgaaagtgactttctgccttcctgaaataagtcaagttcctatgggaggttataaaattatttttgaatatgcaaatcgattgcaagaaaggggacacaaggtaactattgtctttttaacacataatgtgtggagtctactaaccaataacaagaagattaaatctatcgtagggaagattagaggacggaatgaaccatcgtggtttcatttaaattctgaaatacaaaaaataatgacaccttatttagatggtcgagagtttcctgacgcagattttatttttgcaactgcagtaacaacagctaagattgttaaagaattaccatctcgttgtggtaaaaaatgttattttattcaaggttttgaaacatttatccttccagaggatgaagttgttgaatcttatcaatatggttttctaaatttcacagtttcaaattggttgagtaatattgtgggagggtatacaagtgataaaacaatctgccttccaaatcctatagatactgaaaattttaaagtgataactcctataaacgaacgtaatccttacacattggggatgctttatcatgaaggggagcataaaggaattccctacgctttagaagccattgatatagtcaaaaaaaaatataaggatgttgtggttaacatttttggtgtaccgaatcgtccagaatcgttacctgattattttagatatgtacagaaagcaaatcagaatgatttattgagcttatataatgctacttcaatttttatttgtgcaacaattgatgagggctttggtttaacaggcgctgagagtatggcttgtggttgtgcattagtttctacagcttataatggtgttcttgaatatgcagtaaatgccgaaaatgctttattgtctcccgtgagagatagtaaagcattagctgaaaacatcataaaacttatcgaaaatgacccttatcgtcagtcgttagctaaaaaagctagcgaaactataagtgaaagatcatgggataaaacaattcaaaaattagaaacagttttggcaagtgaactagatatgggtagaggttaatatgattagaatagagagagtgataaatttctttttttatatcaatgttgttctgtacatgttttctttgtctaacattccatttttatttcatatggagtttataaatgttataccaactgttttaggattactagcttatatgatgtactattataaggcaagaaaacttccaactaattcaatgccatctttactattgctacttttgtataccttctttgtagtattgttttggaaaaaatttgatatcgattggtcgctagtaagtatattaatatatgttccattaattgagtctgaaaataagggctttatccgtgggcttatacttgttaaattatttgtattaatgattgttgtagtattgtcacttcttggaataataactgataccgtttacttgaaattaggtggtgtcagtcattcacttggtttttttcatcctaatacgttaggagtagtcagtttatctattttctttgactgttttatattatttcaagagttgagaaaacaatattttacattttttcttcttacagtattatacatagtctatcacctaacattttcaagaacatcaattattatagctggactaggattattaatttatgcattccgtaagatattgtcgagttatgtaatgaataagtttgttttctcgattactactactggtgtgtatctttttggaatgttttactcaatgttttatagcacttccaacatcttttataggcaattagatatatttctaactaatagagttcgcctgggaaatatgtatattcaaagatatggttttacactttttccaaaaagtactcccaatatcatacctcaaggatggtggagttcagaccaactttttaatgataatacatttctgaaatttgcacttacacagggagtaattattgctgtattatttctgctatatattttatacattataggaactaaagagtattgcttgtacgatgcgttgcttattttactaacgtttttattcttaatgatcgaagctctaggattttacgccttcttattgacaccattactcttcaagttcatggctgttagagatagtagtgtgaagcaaatataaaaattatcataacatgtttatgcgataaaggtgatttatgaaaacagtaaaaaactatgtttatagttcattatatcaattatttttaatcattgttccatttgcaacaattccttatgtatcacgtgtgttgggtgctgagttgataggtgtcaattcgtttacgaacacaatcatgacatattttgtactattcgcaaacataggaacaactgtttacggaaatcgaacaattgcttattaccgagaatcaataacgaagcgaagtcaaaagttttgggaaatccttattctaaaattggttgtttcattagttatctacctcatgttcttagtttttatttggttataccctaactatagaaccgtttttatgatacaatcagtccaaattttggctacagcagttgatgtctcatggctgtttgatggattggaagatttcaaacgtacagttgttcgtaattttatcgtgaaattggcatctgtaatgatgattttcttgtttgtaaaaactcctcaggattttaatacatatgttttaatcatggtggggtcgtctttagtggggaatctaactctttggacatatttaagacgctatattacaagtattaagtttactaatttgaatatagcagagcattttgcaccagtgttcgctttatttgttccacagattgcctctactatttttgtttccttaaataaagttttgctaggaactctatcaacgatgtcacaggcaggttattttgaaaatgctgataaggtaataagagttttactggcgttagtttcgtctattggtgtagttatttttccaaaggttgctaatgcctataagaatcgagacactaagaaagtgatagaactgacaaagttaacttttaatgctgttaatatcattacaattcctatggtaattgggatgatatcaattagtgatattttttcggatatcttttttggaccggagtttttgggaattgatattgtattgtcaattttaattctagaactattatttatgggatattcttcagtgttgggaagtcaatatttgattgctacaggacaggcaaaatatttaagtgttgctgtgattggcggtctttttactaatagtgtatcgtctattattctgattcctcatcatggtgctgttggggcagcaattgcgtcagtggttggagaggctactatagcgttcatcgagcttatttttatcagaaagcaaatcaatatattagagttatgtaaggacgtccctaaatatagtatagcaagcctattaatgtttttaggaatctatatgacaaagttactcacagtccctccctatataaatttgtttatgagaattatagtaggcggggttatctatggcgtaagtttattgttgctgaagccacaattggtgaaaatttctattgagaaaataaagtttgctaagaaataaatggaaagcagtgcgacgataagaaacgattgtttagattaattttacaagttatgtttaaataaaacatcataatctcaaaactatgggaatagcaaccttaggaattatatcagaatttgcccttatgatggcttagggaaaatatattggctctttgtcaactgtagtgggtagatgaaaagctaaattcttgagaggaccagtttggtcttctcttttttaatatttactgccatcaaaatccgtttcttaaagttttcaaagttcctgaagccaaaggcatttcgcttaatgaccttgatgagattgttcgttgcttccagttttgcgtttgaatacggcagttcgagggcgttgataatcttgtctttatctttcaaaaaagttctaaaaactgtctgaaagatagggtgtacgctggaactcgtctcttcaatgaggtcaaagaaatggtccgagttcttctcttggaaatggaaaagtaaaagttggtagagttcataatgttctctcagttcttgagagtatgacagtagcttttctaggatttccttgttggttaaatgcgcacgaaaagtcggacgataaaagcgtttatcgttgagtttgcgactatcttgttggatcagtttccagtagcgtttcagggcacgatattcctgtgactgacggtccaaatggttcatgatttgaatgcggacgcggttcatagcacgactaaggtgttgcacaatgtgaaagcgatcaaggacaatcttagcgttagggaataattttctggcgatgtcgtaataagggctaaacatgtccatagtgatgactttaacgcgatttctatcctgtctggaatagcgtaggaagtggtttctaatcgttgcttgcgtgcgtccgtcgaggattgtgatgacttttaatgagtcgaaatcctgagcgatgaaactcatttttcccttcttgaagccgtactcatcccagctcatattctcaggcagccaattccagtcggttttaaattggaactcatttagcttgcgactgacggttgaggtagagacggcaaggcgcttggcaatgtcggtcatagactgtttttcaatgagcaattgagcgattttctgatagacgacggttgcgatttggtgattcttcttgaccagagaagtttccgcgacagccatttttccgcactccttacacttgaaacggcgctttttcaggcgaatgagagttcggtagccagcacactccagatacgggattttagaggctttctggaagtcgtatttgcccatctgtcccttgcaggcaggacatttaggagactcgtaatccaagtaaccgtgaagttctttgtgcgttcccatatcgtattcattagtgatgataatatttttgtcttttattccaagaaaatttgtgataagatttagttgttccatatgattctttctaaaatgatggtttagtcgcttttcattataggtcatatgggactttttttctacaatcaaaaaggctccataatctccatagaggatttacccactacagaaattatagagccaatatattgacagggagcttagctccctttttcaaatgacactaatgtttaaacatggtaaaatagacttaataaatttttgattaaaaggtgtcataacatgaaaggtattattcttgcaggtgggtctggaactcgcctttatccactgactcgtgcggcgtcaaaacagttgatgccaatctatgataagccaatgatttactatccgctttcaactttgatgttggctgggattaaagaaatcttgattatttcaactcctcaggatcttcctcgtttcaaggatatgctgggagacggctcagagcttggtatcaccttgtcatacgcagaacaaccaagcccagacggcttggcacaagcctttatcatcggtgaagcctttatcggtgatgacaacgtagccttgattttgggcgacaacatctatcatggaaatggtttgaccaagatgttgcaacgagctgctagcaaagaaaaaggtgcaactgtctttggttaccaagttaaagacccagaacgctttggtgttgtcgagtttgacgcagacatgaacgctatctctatcgaagagaaaccagaagagccaaaatcaaactttgcggttactggtttgtacttctatgacaatgatgttgtggagattgctaaaaacatcaaaccgtcacctcgtggagagttggaaatcaccgatgtcaacaaggcttacttagaccgtggcgacttgtctgtggaactgatgggacgcggattttcttggttggatacaggtacgcatgagagcctcttggaagctgctcaatacatcgaaacggttcaacgtttgcaaaacgttcaagtggcaaacttggaagaaattgcctaccgcatgggctacatcacaaaagaacaagtgtacgaattggcacagccactcaagaaaaatgaatacggacaatacttgctccgtttgattggagaagcttaatgacagaaaactttttcggaaaaacactcgcagcccgtccagttgaaggtattccaggaatgttagaatttgacattccagttcatggcgacaaccgtggctggttcaaggagaacttccaaaaggaaaaaatgtggctccttgtcaactgtagtgggttacgaaaagctaacatctagagaggaccggataggtcctttttttatgtatgttcagtgtgatgaagacacgcttcttaaagttgataaagtttctaaaaccgaagcccaaacgtttgatgtctttaatcaacttgttagtcgcttcaagtttcgcgtttgaatagtccgtttctagtgcgtttttgatgtattgcttgtgtctaagaaaagtcctaaagacagtttgaaaataatgattgaccttgctcctattttcctctatcagttcaaagaaatcatctactctcttctcctgaaagtgaaaaagcaaaagctgataaagtgtatagtagtcagtaagctcttttgaaaagactagtgtcttcgcaacaacttcatgtggtgctaaagtttggcggaaagtctttgaataaaaagaattgagagatagtttacagctgtccttttggaagagtcgccagtgatttttcaaggctcgatagggtagtgacttcttatcgaattggttcatgattgcaattctagtctttaaaaatgctctaccaaggtgctggataatgtggaaacgatcaagaacgattttggcgtttggaaagagtctgcgggatagtgggatataggctccagacatatccatcgtgataaacggcacctgttgtcggactttcaatggatacttcaaaaagtagtttcgtatagtagtttggcggcgattatcaaggatgattatgagttcgtttgtctcatacttttgagctacaaaagccaactctcctttcttgaaaccaaactcatcccaggacataacagtagggagtttgtcataatgttccttgaaagtaaactgatcaagcttacaatagacagtggacgtcgacacgcgaagtcttcttgcaatatcagttagtgacactttctcagttaggagttgtgtaactttttgtcggactagattggagatttggcagtttttctcaacgatagatgtctcagccaccgttactctcctacaatttttacactggaaacgacgttttttcagacgtagtagagttggcgttcccgcttgctcgagaagagggattttagaaggcttttgaaagtcgtacttgatcatctttccatggcaatgaggacatgatggtgcagggtaatcaagttttgcttgaatctcgatatgagtgtcagtttcaaaaacaagtgaaataatgatattttggtctttaattccgattaattatgtggtattcttaataggtctcataagttcttcctaatggtagtttcgtcgcttttcattatagttcttatgggactttttgtctgcattcaaaaagctctataatctctacaatggttttacccactacagaaattatagagccaatatatctactgtctatttttatgctacttttgggttagctcaactcaaccgccttttaatctcccaacaacaataatacccaattaaacaacccaagaaattcaagataatatcactaatggcaaatgtgcccaaataaaagataaattgaatggtttcaattcctaaaagtgtgaccaaactgacaatgacaaactgtttgaaatcagtattgatacagtaaaggccacctaaaggaatgaagtagataatatttagcactgcctcttgaatcgttctggtatccgcttttataaagtcaaaaggattaagtgatatcgcctgaaaatccgttgttttagtaaaaagcaccatgaataacagtaataaatacacactgaaagcaagatagagataaataactgaaaaatgtttgaggtgatactggatgccaaacaaccagataatcagcgttaataagagtattaaagttaatgtggtatagtcaaagtggttaatcaacctagtcaggctttgatagcgagtgagaacgggcataatcagccaagtaatcgtcgcataactcaggataaatgtgaccaataaactgctgaggtagatcatatattttcgcaa

(19) United States
(12) Patent Application Publication
(10) Pub. No.: US 2025/0043033 A1
(43) Pub. Date: Feb. 6, 2025

(54) POLYSACCHARIDES RESULTING IN IMPROVED WATER HOLDING CAPACITY OF DAIRY PRODUCTS

(71) Applicant: Chr. Hansen A/S, Hoersholdm (DK)

(72) Inventors: Vera Kuzina Poulsen, Hoersholm (DK); Paula Gaspar, Hoersholm (DK); Kristian Jensen, Hoersholm (DK); Rute Neves, Hoersholm (DK); Ahmad Zeidan, Hoersholm (DK)

(73) Assignee: Chr. Hansen A/S, Hoersholdm (DK)

(21) Appl. No.: 18/706,846

(22) PCT Filed: Nov. 23, 2022
(86) PCT No.: PCT/EP2022/082940

§ 371 Date: May 2, 2024

(30) Foreign Application Priority Data
Nov. 25, 2021 (EP) 21210446.7

(51) Int. Cl. C08B 37/00; A23C 19/032; A23C 19/068; C12N 1/20; C12R 1/46

(52) U.S. Cl. CPC C08B 37/006; A23C 19/0323; A23C 19/0684; C12N 1/205; A23C 2220/206; C12R 2001/46

(57) ABSTRACT

The present invention provides EPS structures, as identified by their repeating unit, the corresponding eps gene clusters and strains producing such EPS which result in the desired combination of water-holding capacity, texture and acidification speed. The invention further provides methods of using the EPS and strains producing such EPS for producing food product.

### FIELD OF THE INVENTION

The present invention relates to novel exocellular polysaccharides resulting in improved water holding capacity of dairy products and bacterial strains producing such polysaccharides. These polysaccharides and strains also enable retention of the desired shear stress properties and acidification times otherwise difficult to combine with high water holding capacity.

### BACKGROUND OF THE INVENTION

Dairy companies are constantly looking for ways to optimize their production, e.g. by investing in energy optimization, changing their product mix or through better utilization of waste streams. One solution to increase the product yield, i.e. to get more product out of a given amount of milk, is to use starter cultures able to retain more moisture in cheese.

Increased water-holding capacity enables cheesemakers to produce more cheese from the same amount of milk, thus, amongst other advantages, reducing the carbon footprint of their products. As a result, cheese production becomes more sustainable: customers reduce their costs as well as their CO₂ footprint. Thus, increased water-holding capacity ensures decreased syneresis in fermented milk products and improves cheese yield. On industrial scales, even a relatively small percentage improvement in water holding capacity can lead to large savings, both in terms of financial and environmental costs.

The use of polysaccharide producing starter cultures can help increasing the water-holding capacity of fermented milk leading to a decreased level of whey separation (syneresis) in yoghurt and increased moisture content and yield in cheese (Amatayakul et al., 2006).

Consequently, there is an urgent need in the industry for polysaccharides and strains producing such polysaccharides resulting in a high water-holding capacity while at the same time fulfilling all other relevant criteria for commercially useful strains such as texture and acidification speed.

There is an extremely large variety of possible polysaccharide structures (identified by their repeating unit) determined by their sugar building blocks, anomeric configuration, glycosidic linkage, non-sugar decorations of the monosaccharides and conformation.

The selection of suitable polysaccharides and polysaccharide-producing cultures to be used in fermented milk products depends on numerous factors such as functionality of the polysaccharide produced, product type and production process. For example, in the context of the present invention, suitable polysaccharides need to achieve the desired water holding capacity and must also fulfil other criteria in order to be commercially useful. Importantly, polysaccharides that result in high texture are not suitable for the production of cheese such as Mozzarella. Therefore, suitable polysaccharides must combine high water holding capacity with relatively low shear stress to avoid viscous whey during the cheese production process, which is problematic. Additionally, strains producing such polysaccharides must be able to acidify the milk relatively quickly, i.e. be able to reach pH 4.5 quickly during fermentation. Slow fermentation is associated with longer waiting times, higher costs and an increased probability of getting a contamination with not desirable microorganisms.

Exocellular polysaccharides can be produced both as unattached material excreted to the environment, which is often referred to as exopolysaccharide (EPS), or as a capsule attached to the surface of the bacteria referred to as capsular polysaccharides or CPS (Zeidan et al., 2017). CPS are less prone to increase the viscosity of the whey compared to ropy EPS, hence they are more favorable for application in cheese making (Awad et al., 2005; Broadbent et al., 2001) where ropy EPS is less desirable due to increase in whey viscosity. The gene cluster encoding for the enzymes responsible for the production of exocellular polysaccharides is commonly referred to as the eps gene cluster.

The teaching in the art was that excreted EPS are not suitable to achieve the desired combination of high water holding capacity and low viscosity. Instead, CPS should be used (Low et al., 1998, Broadbent et al., 2001). While it is still not completely understood which specific structures of either CPS or EPS can achieve the desired combination of water holding capacity and low viscosity, there was strong motivation to investigate further strains producing CPS and to avoid EPS.

### SUMMARY OF THE INVENTION

The inventors have surprisingly found that strains producing predominantly the excreted EPS structures of the present invention show both the desired high water holding capacity and low shear stress. The inventors have also identified the corresponding eps gene clusters and elucidated the structure of the repeating unit of such polysaccharides, and provide strains producing such exocellular polysaccharides, which combine the desired level of water-holding capacity and texture while retaining a fast acidification speed.

The exocellular polysaccharide structures of the present invention have not yet been reported. Further, the existence of EPS and bacterial strains producing such EPS able to unify all desired properties of water holding capacity, relatively low shear stress and acidification speed was not obvious from the prior art, which incited the search for further strains predominantly producing CPS to achieve the desired result.

The present invention also relates to compositions and starter cultures comprising such exocellular polysaccharides and/or strains as well as to methods of using the exocellular polysaccharides and/or strains for making food products and to food products comprising the exocellular polysaccharides and/or strains.

### BRIEF DESCRIPTION OF THE SEQUENCE LIST

SEQ ID NO:1 sets out the complete sequence of the eps gene cluster of S. thermophilus strain DSM33981 (or a mutant variant thereof).

SEQ ID NO:2 sets out the complete sequence of the eps gene cluster of S. thermophilus strain DSM33982 (or a mutant variant thereof).

### BRIEF DESCRIPTION OF FIGURES

FIG. 1: Repeating unit structure of polysaccharide produced by DSM33981

FIG. 2: Repeating unit structure of polysaccharide produced by DSM33982

### DETAILED DESCRIPTION

### Definitions

All definitions of herein relevant terms are in accordance of what would be understood by the skilled person in relation to the herein relevant technical context.

In the context of the present invention in any of its embodiments, the expression “lactic acid bacteria” (“LAB”) designates food-grade bacteria producing lactic acid as the major metabolic end-product of carbohydrate fermentation. These bacteria are related by their common metabolic and physiological characteristics and are Gram positive, low-GC, acid tolerant, non-sporulating, rod-shaped bacilli or cocci. During the fermentation stage, the consumption of carbohydrate by these bacteria causes the formation of lactic acid, reducing the pH and leading to the formation of a protein coagulum. These bacteria are thus responsible for the acidification of milk and for the texture of the dairy product. The S. thermophilus strains of the present invention are classified as lactic acid bacteria.

By “water holding strain” in the present specification and claims is meant a strain which preferably generates fermented milks having, under the conditions described below and as exemplified in Example 1 herein, a water holding capacity of 85% or higher, more preferably 88% or higher, or 91% or higher, expressed as the percentage of curd left in the sample after centrifugation and whey removal. For example, the water holding capacity can be between 85-100%, 85-95% or 85-90%. The strains exemplified in Example 1 herein have a water holding capacity of 88-91%.

The water holding strains of the of the present invention may be any bacterial strain. The strains of the invention may be an isolated strain, e.g., isolated from a naturally occurring source, or may be a non-naturally occurring strain, e.g., obtained recombinantly. Recombinant strains will differ from naturally occurring strains by at least the presence of the nucleic acid construct(s) used to transform or transfect the mother strain. In a preferred embodiment of the present invention, the water holding strains of the present invention are lactic acid bacteria. In the most preferred embodiment of the present invention, the water holding strains are S. thermophilus strains.

“Water holding capacity” is an important factor for the efficiency of the production of fermented milk products. The texture of fermented milk is dependent on both the bacteria used for fermentation and process parameters. Exocellular polysaccharides produced by bacteria can positively influence water holding capacity.

Several methodologies have been implemented for measuring water holding capacity in fermented milk, such as centrifugation, siphoning, drainage and nuclear magnetic resonance (NMR) (Amatayakul et al., 2006; Gilbert et al., 2020).

Centrifugation used to estimate the water entrapped into the matrix appears to be the most commonly used method for quantifying water holding capacity. The centrifugation method measures the level of whey separated from the collapsed gel as a result of an applied high external force (centrifugal force), i.e. resistance of the gel to compaction.

In the context of the present invention, “water holding capacity” is measured by centrifugation and expressed as the percentage of curd left in the sample after centrifugation and whey removal.

“Texture” is also an important quality factor for fermented milk products and consumer acceptance is often closely linked to texture properties. The texture of fermented milk is dependent on the exocellular polysaccharide structures, the bacteria used for fermentation and process parameters as well as milk composition. In the context of the present invention, the rheological properties (texture) of a fermented milk product, such as viscosity, can be measured as a function of shear stress of the fermented milk product, as described below.

In connection with the present invention, “shear stress” may be measured by the following method: When the pH of the fermented milk (e.g., mammalian- or plant-based milk) reached pH˜4.55 at the incubation temperature e.g. 40° C., the fermented milk product was cooled down by transferring the container to ice water. The fermented milk sample was manually stirred gently by means of a stick fitted with a perforated disc until homogeneity of the sample. The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:
Holding time (to rebuild to somewhat original structure)
5 minutes without any physical stress (oscillation or rotation) applied to the sample.
Oscillation step (to measure the elastic and viscous modulus, G′ and G″, respectively, therefore calculating the complex modulus G*)
   Constant strain=0.3%, frequency (f)=[0.5 . . . 8] Hz
   6 measuring points over 60 s (one every 10 s)
Rotation step (to measure shear stress at 300 1/s)
Two steps were designed:
Shear rate=[0.3-300] 1/s and 2) Shear rate=[275-0.3] 1/s.

Each step contained 21 measuring points over 210 s (on every 10 s). The shear stress at 300 1/s (300 s⁻¹) was chosen for further analysis, as this correlates to mouth thickness when swallowing a fermented milk product.

Strains that are able to acidify milk in about 8 h or less may be referred to as “fast-acidifying” strains, i.e., strains which are able to reach a pH of about 4.55 in 8 h or less, measured as follows: 200 mL semi-fat milk (1.5% fat) is heated to 90° C. for 20 min, followed by cooling to inoculation temperature, and inoculated with 2 mL of an overnight culture of the lactic acid bacterium strain, and left at inoculation temperature until the pH 4.55 is reached. The inoculation temperature is 40° C.

The term “sequence identity” relates to the relatedness between two nucleotide sequences or between two amino acid sequences. For purposes of the present invention, the degree of sequence identity between two nucleotide sequences or two amino acid sequences is determined, for example, using multiple sequence alignment tool Clustal Omega (https://www.ebi.ac.uk/Tools/msa/clustalo/; Sievers et al., 2011) with standard parameters.

In the present context, the terms “strains derived from”, “derived strain” or “mutant” should be understood as a strain derived from a strain of the invention by means of, e.g., genetic engineering, radiation and/or chemical treatment, and/or selection, adaptation, screening, etc. It is preferred that the derived strain is a functionally equivalent mutant, e.g., a strain that has substantially the same, or improved, properties with respect to water holding capacity as the mother strain. Such a derived strain is a part of the present invention. Especially, the term “derived strain” or “mutant” refers to a strain obtained by subjecting a strain of the invention to any conventionally used mutagenizing treatment including treatment with a chemical mutagen such as ethane methane sulphonate (EMS) or N-methyl-N′-nitro-N-nitroguanidine (NTG), UV light or to a spontaneously occurring mutant.

A mutant may have been subjected to several mutagenizing treatments (a single treatment should be understood as one mutagenizing step followed by a screening/selection step), but it is presently preferred that no more than 20, no more than 10, or no more than 5, treatments are carried out. In a presently preferred derived strain, less than 1%, or less than 0.1%, less than 0.01%, less than 0.001% or even less than 0.0001% of the nucleotides in the bacterial genome have been changed (such as by replacement, insertion, deletion or a combination thereof) compared to the mother strain.

The terms “fermented milk” and “dairy” are used interchangeably herein. In the context of the present invention in any of its embodiments, the expression “fermented milk product” means a food or feed product wherein the preparation of the food or feed product involves fermentation of a milk base with a lactic acid bacterium. “Fermented milk product” as used herein includes but is not limited to products such as thermophilic fermented milk products or mesophilic fermented milk products. The term “thermophilic fermentation” herein refers to fermentation at a temperature above about 35° C., such as between about 35° C. to about 45° C. The term “mesophilic fermentation” herein refers to fermentation at a temperature between about 22° C. and about 35° C.

In the context of the present application, the term “milk” is broadly used in its common meaning to refer to liquids produced by the mammary glands of animals (e.g., cows, sheep, goats, buffaloes, camel, etc.) or produced using plant bases. The term “milk base” or “milk substrate” may be any milk material that can be subjected to fermentation according to the present invention. Thus, useful milk bases include, but are not limited to, solutions/-suspensions of any milk or milk like products comprising protein, such as whole or low-fat milk, skim milk, buttermilk, reconstituted milk powder, condensed milk, dried milk, whey, whey permeate, lactose, mother liquid from crystallization of lactose, whey protein concentrate, cream, or plant-based milks. The milk base may originate from any mammal, e.g., being substantially pure mammalian milk, or reconstituted milk powder. Plant sources of milk include, but are not limited to, milk extracted from soybean. Preferably, the plant-based milk is soy milk, which can be preferably supplemented with glucose, such as 0.5-5% glucose, preferably 0.5-2% glucose, more preferably about 2% glucose.

Prior to fermentation, the milk base may be homogenized and pasteurized according to methods known in the art.

“Homogenizing” as used in the context of the present invention in any of its embodiments, means intensive mixing to obtain a soluble suspension or emulsion. If homogenization is performed prior to fermentation, it may be performed to break up the milk fat into smaller sizes so that it no longer separates from the milk. This may be accomplished by forcing the milk at high pressure through small orifices.

“Pasteurizing” as used in the context of the present invention in any of its embodiments, means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the context of the present invention in any of its embodiments means the conversion of carbohydrates into acids or alcohols or a mixture of both-through the action of microorganisms (LAB). Fermentation processes to be used in production of food products such as dairy products are well known and the person of skill in the art will know how to select suitable process conditions, such as temperature, oxygen, amount of microorganism(s) and process time. Fermentation conditions are selected so as to support the achievement of the present invention, e.g., to obtain a food product, preferably a food product which has an improved water holding capacity as compared to a food product produced with a method which does not involve the use of at least one of the EPS structures and/or strains producing such structures as described in the first aspect of the present invention or the use of the composition as described in the second aspect of the present invention, in any of its embodiments.

The term “dairy product” as used herein refers to a food product produced from milk. As described above, in the context of the present application, the term “milk” is broadly used in its common meaning to refer to liquids produced by the mammary glands of animals (e.g., cows, sheep, goats, buffaloes, camel, etc.) or by plants. In a preferred embodiment, the milk is cow's milk. In accordance with the present invention the milk may have been processed and the term “milk” includes whole milk, skim milk, fat-free milk, low fat milk, full fat milk, lactose-reduced milk, or concentrated milk. Fat-free milk is non-fat or skim milk product. Low-fat milk is typically defined as milk that contains from about 1% to about 2% fat. Full fat milk often contains 2% fat or more. The term “milk” is intended to encompass milks from different mammals and plant sources. Mammalian sources of milk include, but are not limited to cow, sheep, goat, buffalo, camel, llama, mare and deer. Plant sources of milk include, but are not limited to, milk extracted from soybean, pea, peanut, barley, rice, oat, quinoa, almond, cashew, coconut, hazelnut, hemp, sesame seed and sunflower seed. In a specific embodiment, the milk is cow's milk. In another specific embodiment, the milk is a plant-based milk, preferably soy milk, which can be preferably supplemented with glucose, e.g., 0.5-5% glucose, preferably 0.5-2% glucose, more preferably about 2% glucose.

As used herein, the term “about” (or “around”) means the indicated value ±1% of its value, or the term “about” means the indicated value ±2% of its value, or the term “about” means the indicated value ±5% of its value, the term “about” means the indicated value ±10% of its value, or the term “about” means the indicated value ±20% of its value, or the term “about” means the indicated value ±30% of its value; preferably the term “about” means exactly the indicated value (±0%).

Throughout the description and claims the word “comprise” and variations of the word (e.g., “comprising”, “having”, “including”, “containing”) typically is not limiting and thus does not exclude other features, which may be for example technical features, additives, components, or steps. However, whenever the word “comprise” is used herein, this also includes a special embodiment in which this word is understood as limiting; in this particular embodiment the word “comprise” has the meaning of the term “consist of”.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The exocellular polysaccharide structures and eps gene clusters of S. thermophilus strains It is an object of the present invention to provide a structure of exocellular polysaccharide produced by S. thermophilus strains, which contributes to improved water holding capacity suitable for use in preparation of food products. The inventors have also analyzed the corresponding eps gene cluster and identified gene sequences that are involved in the production of the exocellular polysaccharide of the present invention. These exocellular polysaccharide are involved in the excellent water holding capacity and relatively low shear stress desirable in the production of cheese such as Mozzarella. As shown in Example 1, strains producing such exocellular polysaccharides (DSM33981 and DSM33982) yield fermented milk with excellent water holding capacity.

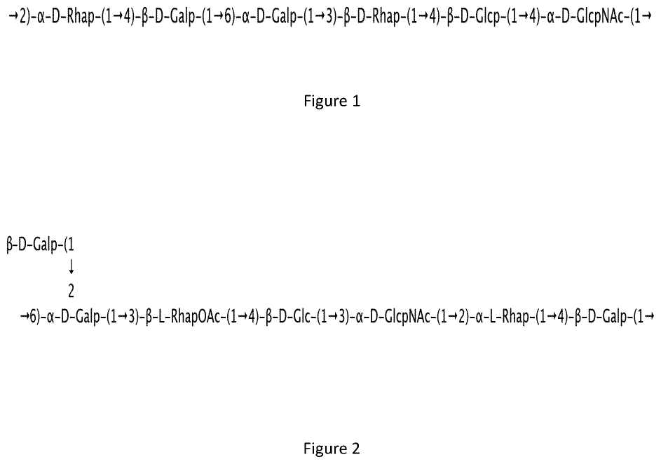

Strain DSM33981 has the eps gene cluster of SEQ ID NO.: 1 produces polysaccharides with a repeating unit as shown in FIG. 1. Strain DSM33982 has the eps gene cluster of SEQ ID NO.: 2 produces polysaccharides with a repeating unit as shown in FIG. 2.

Strains producing these polysaccharides preferably have a sequence identity of 95% or more to the listed eps gene clusters, such as 95%, 96%, 97%, 98% or 99%. Most preferably, the sequences have 100% identity.

LAB have been shown to produce exocellular polysaccharides through two pathways, encoded by distinct genomic components: the sucrase-dependent pathway and the Wzy-dependent pathway (Zeidan et al., 2017). S. thermophilus do not have sucrose dependent pathway, and synthesize exocellular polysaccharides only through the Wzy-dependent pathway, which is encoded in a gene cluster, here referred to as an eps cluster, (Zeidan et al., 2017). Generally, eps clusters consist of regions of well-conserved genes flanking a variable region of biosynthetic genes encoding glycosyltransferases. This variability is what underlies the diversity of polysaccharide structures, as the glycosyltransferases are responsible for building the repeating units that are subsequently assembled into long polymers. The repeating unit is synthesized intracellularly, with the nascent glycoside anchored to the lipid membrane. The first step of the process is catalyzed by the priming glycosyltransferase, which primes the lipid anchor, usually undecaprenyl-phosphate, by attaching a sugar monomer to it (Islam et al., 2014). After the first sugar monomer is attached, glycosyltransferases encoded in the eps cluster sequentially add the remaining sugar monomers until the entire repeating unit has been completed.

When the repeating unit has been completed, it is flipped to the outside of the cell membrane and added to an elongating chain of repeating units by a polymerase (Islam et al., 2014).

This structure is believed to have an impact on the differences in the water holding capacity of the different LAB strains.

The Compositions Comprising the Exocellular Polysaccharide and/or S. thermophilus Strains Producing Such Exocellular Polysaccharide

The present invention also provides compositions and starter cultures comprising the exocellular polysaccharide and/or the S. thermophilus strains of the invention as described. LAB, including bacteria of the species S. thermophilus, are normally supplied to the dairy industry either as frozen (F-DVS®) or freeze-dried (FD-DVS®) cultures for bulk starter propagation or as so-called “Direct Vat Set” (DVS®) cultures, intended for direct inoculation into a fermentation vessel or vat for the production of a dairy product, such as a fermented milk product. Such lactic acid bacterial cultures are in general referred to as “starter cultures” or “starters”. Accordingly, the composition of the present invention may be frozen or freeze-dried. In addition, the composition of the present invention may be provided in liquid form. Thus, in one embodiment, the composition is in frozen, dried, freeze-dried or liquid form.

The compositions or starter cultures of the present invention may also additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the compositions or starter cultures according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity, e.g., cultures may lose their acid-producing (acidification) activity when kept stored even for shorter periods of time. Thus, viability and booster effect have to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i.e. the production of, e.g., lactic acid, acetic acid, formic acid and/or propionic acid, or its metabolite producing activity such as the production of aroma compounds such as acetaldehyde, (α-acetolactate, acetoin, diacetyl and 2,3-butylene glycol (2,3-butanediol)).

In one embodiment the compositions or starter cultures of the invention contains or comprises from 0.2-20% of the cryoprotective agent or mixture of agents measured as % w/w of the material. It is, however, preferable to add the cryoprotective agent or mixture of agents at an amount which is in the range from 0.2-15%, from 0.2-10%, from 0.5-7%, and from 1-6% by weight, including within the range from 2-5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In a further aspect, the compositions or starter cultures of the present invention contains or comprises an ammonium salt (e.g. an ammonium salt of an organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH₃), NH₄OH, NH₄⁺, and the like.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

### Method for Producing Food or Feed Products

The present invention further relates to methods of producing a food product comprising at least one stage in which at least one exocellular polysaccharide and/or S. thermophilus strain as defined in the first aspect of the present invention and/or the composition or starter culture as defined in the second aspect of the present invention are used. The production of the food product is carried out by methods known to the person skilled in the art.

In one aspect, the present invention relates to a method of producing a food product comprising at least one stage in which at least one of the exocellular polysaccharides of the present invention are used.

In another embodiment, the present invention relates to a method of producing a food product comprising at least one stage in which the lactic acid bacterium strain S. thermophilus DSM33981 or a mutant or variant therefrom is used.

In another embodiment, the present invention relates to a method of producing a food product comprising at least one stage in which the lactic acid bacterium strain S. thermophilus DSM33982 or a mutant or variant therefrom is used.

For instance, the method of the present invention comprises fermenting a milk substrate with a composition comprising at least 1×10⁶ CFU/mL, preferably at least 1×10⁸ CFU/mL of strain S. thermophilus DSM33981 or a mutant or variant therefrom.

For instance, the method of the present invention comprises fermenting a milk substrate with a composition comprising at least 1×10⁶ CFU/mL, preferably at least 1×10⁸ CFU/mL of strain S. thermophilus DSM33982 or a mutant or variant therefrom.

In another preferred embodiment, the method comprises fermenting a milk substrate with the composition as described in the second aspect of the present invention, in any of its embodiments.

Preferably, the food product is a dairy product and the method in any of its embodiments comprises fermenting a milk substrate (also referred to as “milk base” in the context of the present invention) with the at least one S. thermophilus and/or with the composition or starter culture according to the invention.

Preferably, the food product is a dairy product and the method in any of its embodiments comprises fermenting a plant-based milk substrate (also referred to as “plant-based milk base” in the context of the present invention), such as soy milk, preferably soy milk supplemented with glucose, e.g., with 0.5-5% glucose, preferably 0.5-2% glucose, more preferably about 2%, with the at least one S. thermophilus strain and/or with the composition or starter culture according to the invention (first and second aspects, respectively).

The food product according to the present invention may advantageously further comprise a “thickener” and/or a “stabilizer”, such as pectin (e.g. HM pectin, LM pectin), gelatin, CMC, Soya Bean Fiber/Soya Bean Polymer, starch, modified starch, carrageenan, alginate, and guar gum.

In a specific embodiment the food product is a dairy product, a meat product, a vegetable product, a fruit product or a cereal product. In a preferred embodiment, the food product is a dairy product, as defined above. In another preferred embodiment, the food product is a plant-based food product, such as fermented soy milk.

In a particular embodiment of the invention, the fermented milk product is selected from the group consisting of Mozzarella cheese, yoghurt, kefir, sour cream, cheese, quark. Mozzarella is particularly preferred. In a preferred embodiment of the invention, the fermented milk product contains a further food product selected from the group consisting of fruit beverage, cereal products, fermented cereal products, chemically acidified cereal products, soymilk products, fermented soymilk products and any mixture thereof. In another preferred embodiment, the fermented milk product is a plant-based fermented milk product, such as fermented soy milk.

The fermented milk product typically contains protein in a level of between 1.0-12.0% by weight, preferably between 2.0-10.0% by weight. In a particular embodiment, sour cream contains protein in a level of between 1.0-5.0% by weight, preferably between 2.0-4.0% by weight. In a particular embodiment, Quark contains protein in a level of between 4.0-12.0% by weight, preferably between 5.0-10.0% by weight.

Preferably, the food product has an improved water holding capacity (as described in the present invention such as in Example 1) as compared to a food product produced with a comparable method which does not involve the use of at least one of the EPS, S. thermophilus strain with an active eps gene cluster as described in the present invention and/or the use of the composition or starter culture as described.

Any combination of the above-described elements, aspects and embodiments in all possible variations thereof is encompassed by the invention unless otherwise indicated herein or otherwise clearly contradicted by context. Embodiments of the present invention are described below, by way of examples only.

### EXAMPLES

### Example 1: Evaluation of Water Holding Capacity of Fermented Milk Using S. thermophilus Strains Producing the Desired Exocellular Polysaccharide

Cultures incubated overnight in M17 broth containing 2% lactose were used for milk acidification (1% inoculum) in Severely Heat-Treated skim milk (SHT-SM) for shear stress measurement and pasteurized low-fat milk (past-LFM) for water holding capacity measurements (e.g. for Mozzarella cheese application). SHT-SM was prepared by reconstituting skim milk powder containing 38% protein, 53% lactose, S. thermophilus (Derzelle, Bolotin, Mistou, & Rul, 2005).

Water-holding capacity was measured by centrifugation and expressed as the percentage of curd left in the sample after centrifugation and whey removal.

Pasteurized low-fat milk from Arla containing 1.5% fat and 3.8% protein was pasteurized at 90° C. for 20 min, cooled down to the 40° C., and enriched with 0.003% Na⁺-formate (HCOONa) to stimulate the growth of S. thermophilus.

Empty sterile plastic bottles were weighed, and 200 ml pasteurized low-fat milk containing 1% overnight culture and 0.003% Nat-formate was added. The samples were incubated at 40° C. until a pH of 4.55 and subsequently cooled down in ice water for 30 min and then kept overnight at 4° C. The samples were centrifuged at 2325*g for 3 min, and the whey was carefully poured out of the plastic bottles. The samples were weighed before and after whey removal. The water holding capacity (WHC) was expressed as percentage of curd left in the sample after whey removal. Four replicates of each sample were prepared; one was used to monitor milk acidification using pH electrodes, and three remaining replicates for water holding capacity measurement.

TABLE 1
Water holding capacity (WHC), shear stress at shear
rate 300 1/s, time to pH 4.55 of fermented milk
using strains producing the EPS of the invention.

| | WHC | Shear stress | Time-to-pH |
|---|---|---|---|
| Strain | (% of curd) | at 300 1/s (Pa) | 4.55 (hour) |
| DSM33981 | 91 | 23.1 | 8 |
| DSM33982 | 89 | 17.6 | 5 |

The two example strains with the desired eps gene cluster producing the claimed exocellular polysaccharide structures clearly show a combination of high water holding capacity and low shear stress.

### Example 2: Evaluation of EPS and CPS Production in S. thermophilus Strains

Polysaccharide fractions were purified from samples collected in stationary phase of growth by ethanol precipitation and quantified by the phenol: sulfuric acid method to determine total sugar content. Growth was carried out in Chemically Defined Medium supplemented with 2% Lactose (CDMLac) at constant pH of 6.5 and 40° C. under anaerobic conditions. pH was kept constant by the addition of NaOH. Values are the average of at least 5 technical replicates replicates±standard deviation.

TABLE 2
EPS and CPS levels and normalized EPS and
CPS levels of the strains of the invention.

| | EPS and CPS levels | | | | | | | |
|---|---|---|---|---|---|---|---|---|
| | normalized by biomass (OD₆₀₀) | | | | EPS and CPS levels | | | |
| | EPS | | CPS | | EPS | | CPS | |
| | (μg/mL/OD₆₀₀) | | (μg/mL/OD₆₀₀) | | (μg/mL) | | (μg/mL) | |
| | average | stdev | average | stdev | average | stdev | average | stdev |
| DSM33981 | 8.4 | 0.5 | 3.7 | 0.2 | 36.7 | 2.4 | 16.1 | 0.8 |
| DSM33982 | 7.8 | 0.2 | 1.3 | 0.1 | 35.3 | 0.8 | 5.7 | 0.3 |

The example strains predominantly produce the excreted EPS of the present invention. It was previously thought that higher amounts of EPS would necessarily lead to concomitant increase in viscosity, i.e. higher shear stress. EPS with a structure as claimed are able to unify both high water holding capacity and relatively low shear stress (Example 1).

### Example 3: Determination of Exocellular Polysaccharide Structure

Strains were grown in a Chemically Defined Medium under anaerobic conditions for 24 h at 40° C. The culture was then centrifuged (10,000×g, 1 h, 4° C.) and the supernatant was treated with trichloroacetic acid 80% (w/v) to a final concentration of 20%. The precipitation was allowed to proceed at 4° C. with agitation for 2 h. The mixture was centrifuged (10,000 g, 1 h, 4° C.) and the crude EPS was precipitated by the addition to the supernatant of an equal volume of acetone (4° C.). After allowing for overnight precipitation at 4° C. with mild agitation, the sample was centrifuged (10,000 g, 1 h, 4° C.). The pellet was washed three times with 50% acetone (4° C.) and the resulting pellet was suspended in 50 ml distilled water with vigorous agitation. The suspension was subjected to sonication for 5 min leading to a clear solution. Finally, this solution was dialyzed for 12 h against distilled water (5 L) at 4° C. The dialysis was repeated a total of three times, each against fresh distilled water. The content of the dialysis bag was freeze dried and the EPS residue was solubilized in 1 mL D2O by vigorous agitation and sonication.

For the structural elucidation by NMR, the spectra were acquired on an AVANCE III 800 spectrometer (Bruker, Rheinstetten, Germany) working at a proton operating frequency of 800.33 MHz equipped with a three-channel inverse detection probe (TXI) with pulse-field gradients. Two-dimensional spectra for structural elucidation were acquired at 60° C. using standard Bruker pulse programs. Proton-homonuclear shift correlation spectroscopy (COSY), total correlation spectroscopy (TOCSY), ¹H-¹³C multiplicity edited heteronuclear single quantum coherence spectra (ed-HSQC), and two-dimensional HSQC-TOCSY were used for the complete assignment of all the resonances in the HSQC maps. ³J_{H1,H2} as measured directly from the 1D ¹H spectra and ¹J_{C,H1} as measured from non-decoupled HSQC spectra were used to establish the configurations of the sugar rings. Heteronuclear multiple bond connectivity spectra (HMBC) were acquired to determine the position of glycosidic bonds. The identity of the sugar monomers was established by ¹H and ¹³C chemical shift patterns and, when possible, by the determination of distinct ³J_{H,H}.couplings.

TABLE 3
Exocellular polysaccharide structures of the present invention.

| | |
|---|---|
| S. thermophilus (DSM33981) | 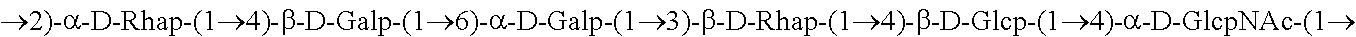 |
| S. thermophilus (DSM33982) | 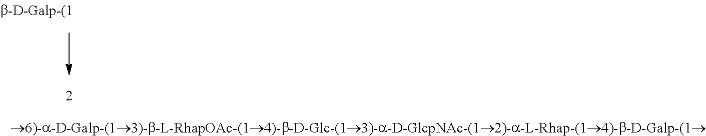 |

### Example 4: Determination of Eps Gene Cluster

As eps clusters have conserved genes at both ends, they were identified using BLAST, by searching translated gene sequences of the annotated genome for the known protein sequences of the first and last gene of the cluster. The organization of genes within the eps clusters in LAB, as reviewed by Zeidan et al. (2017), were used to determine the first and last eps genes in each individual species, e.g. epsA and orf14.9 in S. thermophilus. If a single hit with over 90% sequence identity and over 90% coverage was found for both the first and the last gene, all genes between and including the two hits were extracted and used as the eps cluster.

### DEPOSITS

The strain Streptococcus thermophilus DSM33981 has been deposited at DSMZ (Deutsche Sammlung von Mikroorganismen und Zellkulturen GmbFI, Inhoffenstr. 7B, D-38124 Braunschweig, Germany) under the accession number DSM33981 on Aug. 18, 2021.

The strain Streptococcus thermophilus DSM33982 has been deposited at DSMZ (Deutsche Sammlung von Mikroorganismen und Zellkulturen GmbFI, Inhoffenstr. 7B, D-38124 Braunschweig, Germany) under the accession number DSM33982 on Aug. 18, 2021.

The deposits have been made under the conditions of the Budapest Treaty on the International Recognition of the Deposit of Microorganisms for the Purposes of Patent Procedure. The Applicant requests that a sample of the deposited microorganisms should be made available only to an expert approved by the Applicant.

### REFERENCES

Amatayakul, T., Sherkat, F. and Shah, N. P. (2006). Syneresis in set yoghurt as affected by EPS starter cultures and level of solids. International Journal of Dairy Science, 59, 3, 216-221. https://doi.org/10.1111/j.1471-0307.2006.00264.x
Awad, S., Hassan, A. N. and Muthukumarappan, K. (2005). Application of Exopolysaccharide-Producing Cultures in Reduced-Fat Cheddar Cheese: Texture and Melting Properties. Journal of Dairy Science, 88, 12, 4204-4213. https://doi.org/10.3168/jds.S0022-0302 (05) 73106-4
Broadbent, J. R., McMahon, D. J., Oberg, C. J. and Welker, D. L. (2001). Use of exopolysaccharide-producing cultures to improve the functionality of low fat cheese. International Dairy Journal, 11, 4-7, 433-439. https://doi.org/10.1016/S0958-6946 (01) 00084-X
Gilbert, A., Rioux, L. E., St-Gelais, D., and Turgeon, S. L. (2020). Characterization of syneresis phenomena in stirred acid milk gel using low frequency nuclear magnetic resonance on hydrogen and image analyses. Food Hydrocolloids. 106. https://doi.org/10.1016/j.foodhyd.2020.105907
Islam, S. T., and Lam, J. S. (2014). Synthesis of bacterial polysaccharides via the Wzx/Wzy-dependent pathway. Canadian Journal of Microbiology, 60(11), 697-716. https://doi.org/10.1139/cjm-2014-0595
Low, D., Ahlgren, J. A., Horne, D., McMahon, D. J., Oberg, C. J., and Broadbent, J. R. (1998). Role of Streptococcus thermophilus MR-1C capsular exopolysaccharide in cheese moisture retention. Applied Environmental Microbiology, 64, 6, 2147-2151. https://doi.org/10.1128/AEM.64.6.2147-2151.1998
Sievers, F., Wilm, A., Dineen, D., Gibson, T., Karplus, K., Weizhong, L., Lopez, R., McWilliam, H., Remmert, M., Söding, J., Thompson, J., and Higgins, D. G. (2011). Fast, scalable generation of high-quality protein multiple sequence alignments using Clustal Omega. Mol. Syst. Biol., 7:539. https://doi.org/10.1038/msb.2011.75
Zeidan, A. A., Poulsen, V. K., Janzen, T., Buldo, P., Derkx, P. M. F., Øregaard, G., and Neves, A. R. (2017). Polysaccharide production by lactic acid bacteria: from genes to industrial applications. FEMS Microbiology Reviews, 41 (1), S168-S200.
https://doi.org/10.1093/femsre/fux017

1. A polysaccharide with a repeating unit structure in which the main chain consists of rhamnose, galactose, glucose and N-acetyl-glucosamine in a ratio of 2:2:1:1, wherein rhamnose is optionally O-acetylated.

2. The polysaccharide of claim 1 in which the main chain of the repeating unit structure is a linear hexamer, optionally containing at least one side branch with at least one sugar.

3. The polysaccharide of claim 1 having a repeating unit structure of:

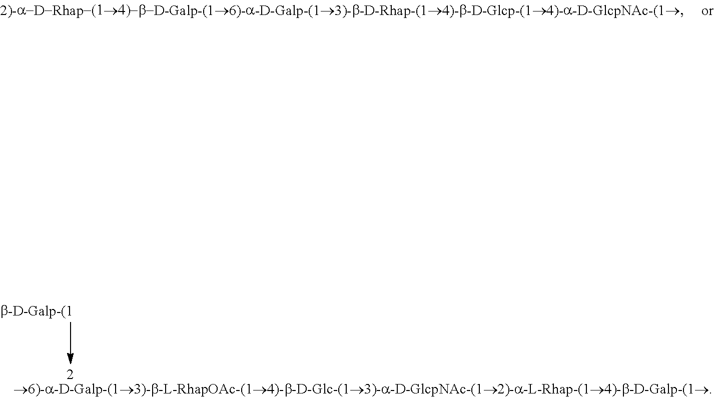

4. A lactic acid bacterium strain comprising an active eps gene cluster capable of producing the polysaccharides of claim 1, wherein the eps gene cluster has a sequence identity of at least 95% with SEQ ID NO: 1 or SEQ ID NO 2.

5. The lactic acid bacterium of claim 4, wherein the bacterium is a S. thermophilus bacterium.

6. The lactic acid bacterium of claim 5, wherein the bacterium is selected from:
(i) DSM33981,
(ii) DSM33982,
or mutant variants thereof with retained or further improved water holding capacity.

7. A composition comprising the polysaccharide of claim 1 and/or a lactic acid bacterium comprising an active eps gene cluster capable of producing said polysaccharide, wherein the eps gene cluster has a sequence identity of at least 95% with SEQ ID NO: 1 or SEQ ID NO 2.

8. A starter culture comprising the polysaccharide of claim 1 and/or a lactic acid bacterium comprising an active eps gene cluster capable of producing said polysaccharide, wherein the eps gene cluster has a sequence identity of at least 95% with SEQ ID NO: 1 or SEQ ID NO 2.

9. (canceled)

10. A method of producing a food or feed product comprising at least one stage in which the polysaccharide of claim 1 and/or a lactic acid bacterium comprising an active eps gene cluster capable of producing said polysaccharide, wherein the eps gene cluster has a sequence identity of at least 95% with SEQ ID NO: 1 or SEQ ID NO 2.

11. The method of claim 10 wherein the food product is cheese such as Mozzarella, yoghurt, kefir, sour cream, or quark.

12. A food or feed product comprising the polysaccharide of claim 1 and/or a lactic acid bacterium comprising an active eps gene cluster capable of producing said polysaccharide, wherein the eps gene cluster has a sequence identity of at least 95% with SEQ ID NO: 1 or SEQ ID NO 2.

13. The food or feed product of claim 12, wherein the food or feed product is a dairy product comprising fermented plant-based and/or mammalian milk.

14. The food or feed product of claim 13, wherein the food product is Mozzarella cheese.

15. A method for manufacturing further lactic acid bacterium strains as defined in claim 4; wherein the mutant strain is obtained by using the deposited strain as a starting material, screening the eps gene cluster and choosing a bacterium which has retained the eps gene cluster.
